(19) United States
(12) Patent Application Publication
(10) Pub. No.: US 2026/0263261 A1
(43) Pub. Date: Sep. 10, 2026

(54) External Catheter for Male Patients and Method of Use

(71) Applicant: Melanie D. Ceraso, Baden, PA (US)

(72) Inventor: Melanie D. Ceraso, Baden, PA (US)

(21) Appl. No.: 19/074,270

(22) Filed: Mar. 7, 2025

(51) Int. Cl. A61F 5/453

(52) U.S. Cl. CPC A61F 5/453

(57) ABSTRACT

An external catheter for male patients in hospital comprises a cross-shaped pad that would frame the patient's shaft or scrotum before being folded onto itself. The pad includes a suction catheter for attaching to wall suction. It would need replacing as often as typical external catheters but proves more effective for male patients who are incontinent.

### CROSS-REFERENCE TO RELATED APPLICATIONS

Not Applicable.

### STATEMENT REGARDING FEDERALLY SPONSORED RESEARCH OR DEVELOPMENT

Not Applicable.

### THE NAMES OF THE PARTIES TO A JOINT RESEARCH AGREEMENT

Not Applicable.

### INCORPORATION-BY-REFERENCE OF MATERIAL SUBMITTED ON A COMPACT DISC OR AS A TEXT FILE VIA THE OFFICE ELECTRONIC FILING SYSTEM (EFS-WEB)

Not Applicable.

### STATEMENT REGARDING PRIOR DISCLOSURES BY THE INVENTOR OR A JOINT INVENTOR.

Not Applicable.

### BACKGROUND OF THE INVENTION.

(1) Field of the Invention. This invention relates to catheters, particularly external catheters. More specifically, it relates to improvements in the field of external urinary catheters for male hospital patients, universal or individually sized.

The catheter includes a three-layered pad that would frame and/or envelope the patient's shaft or scrotum by folding from three sides onto itself. It can serve as a stand-alone unit, or connect via a suction catheter for attaching to wall suction. When connected, it should be noted that the tubing proper runs from the bottom of the fully installed unit rather than from a top/upper edge as is the case with some prior art/competitive products. Once installed, the unit can serve as protective means for eventual replacement as often as typical catheters, preferably every twelve (12) hours unless accidentally soiled. With the present preferred embodiments, this unit is less prone to leakage about the patient's scrotum and urethra, or becoming accidentally discharged when the patient is turned on his side by nursing staff. The device includes an absorbent area, e.g., of a mesh fabric, that will situate below the creases or folds of the uppermost layer. It an also accommodate patients with scrotal edema.

A proposed name for this product is AlphaFit™.

(2) Description of Related Art including information disclosed under 37 CFR 1.97 and 1.98

This invention represents an improvement over the Urological Pad concept shown and described in Russian Application Serial No. RU201701596.

There are known devices intended for the patients targeted by the present invention. For instance, Stryker makes and sells a unit under the name PrimoFit™. A sister product manufactured for female patients is sold under the brand name PrimaFit™

This invention acknowledges numerous issues/concerns with the PrimoFit that it expressly aimed to address and improve upon. See especially accompanying FIG. 1.

Unlike the standard use of PrimoFit, the present device:
1) exhibits no kinking at the drainage bag/suction joint which can lead to overfill and leaking;
2) can fit any size of male genitalia;
3) will NEVER leak when rolling a patient;
4) reduces risk of skin break down at contact site;
5) can be used with or without an adult diaper;
6) can reduce the risk of infection;
7) offers multidirectional wicking to reduce or eliminate moisture exposure;
8) uses less plastic to make and at a fraction of the cost to produce;
9) is nurse tested and approved, far and away more effective than the PrimoFit apparatus as reviewed by floor staff nurses; and
10) is easier to apply and manage than the PrimoFit.

### BRIEF SUMMARY OF THE INVENTION

This invention relates to an improved male catheter consisting of a central pad component with a plurality of adsorption layers, said pad component including a first wing end, a second wing end, a notched recess between the first and second wing end and an apron-shaped base below the first and second wing ends, said pad component adapted for positioning a male patient's scrotum with the notched recess before folding the first and second wing ends and the apron-shaped base about the scrotum. Preferred embodiments include a mesh fabric within the apron-shaped base, an aerated sponge adjacent the mesh fabric and a section of tubing having a plurality of apertures, said tubing section adapted for connecting to wall suction as needed. The first and second wing ends differ in relative lengths so that a longer wing end can overlap a shorter wing end when installed about a patient's scrotum. Adhesive tape means may be included about a perimeter edge of each pad component.

The invention further relates to a method of using such devices for a male hospital patient. The method includes positioning the patient's scrotum within the notched recess of the pad component, folding the shorter wing end about the scrotum and towards a central region of the male catheter, folding the longer wing end over the shorter wing end, folding the apron-shaped base upward to create a pocketed area for the scrotum and affixing the male catheter altogether. If desired, a section of apertured tubing within the apron-shaped base may then be connected to wall suction for the patient.

Problem Solved: Many male patients are not able to use the male external catheter due to the size or retraction of their penis. The current apparatus that is in use is similar to a plastic bag in a funnel shape but efficacy largely depends on size and if bag does not fold over on itself. See generally, FIG. 1—Prior Art. Particularly therein, the prior art male catheter includes a main body B for wrapping about the shaft of a patient, with a T-shaped top adhesion section T, a single layer adsorbent pad framed in plastic P from the bottom of which extends a section of suction tubing S.

As previously discussed, the prior art male external catheter now in use is only effective for larger sized penises and loses its effectiveness at urine collection provided the patient does not move about too much so as to dislodge the application pads or fold off the bag onto itself. These prior art catheters are also known to block with the flow of urine to the suction collector leading to patient/bedding soiling, skin irritations and possibly even urinary tract infection (or UTI) issues.

The present device has proven to be more effective for male patients who experience incontinence while in the hospital. It is a cleaner option than the existing known catheters which are much easier for the nursing staff to manage.

### BRIEF DESCRIPTION OF THE SEVERAL VIEWS OF THE DRAWING(S)

Further features, advantages and objectives of this invention will become clearer with respect to the following description of preferred embodiments made with reference to the accompanying photographs in which:

FIG. 1—PRIOR ART—is a top plan view of a typical Stryker Sage PrimoFit device;

FIG. 2 is a top plan view of male catheter, generally 10, with its the cross-shaped pad component 12 having a first shorter wing end 14, a longer second wing end 16, a notched region between 18 and an apron-shaped base 20 into which is contained a mesh fabric element 22 adjacent an (optional) aerated sponge element 24. Mesh fabric element 22 sits below a centrally located crease 26 in apron-shaped base 20. A short section of tubing 28 (having a plurality of apertures, not seen) extends outwardly from the apron-shaped base 20 for connecting to wall section after installation of the catheter 10 about the male patient's scrotum;

FIG. 3A is a top plan view of the catheter 10 from FIG. 2 showing proposed relative dimensions for a standard size unit;

FIG. 3B is a top plan view of FIG. 2 with proposed dimensions for the larger, bariatric sized unit, roughly three inches longer along main directions;

FIG. 4 is an enlarged top plan view showing the padded elements making up the sub-regions of pad component 12. Particularly, from the views at FIGS. 4 through 6, there is an upper wing layer 30 (FIG. 4), hinged at juncture 32 to a middle/internal layer 34 (FIG. 5) beneath which sits the third of three individualized adsorbent elements, bottom layer 36. Note, that the section of tubing 28 extending outwardly from the catheter's apron-shaped base 20 can be seen partially protruding in FIG. 6;

FIG. 5 is a top plan view of a representative catheter 10 showing its upper wing layer 30 flipped up along hinged juncture 32 to reveal the middle/internal layer 34 below same;

FIG. 6 is a top plan view of bottom layer 36 and section of suction tubing 28 extending downwardly therefrom;

FIG. 7 is a top plan view showing the catheter 10 labeled with numbers to show the preferred order of folding component parts about the scrotum of a male hospital patient from FIGS. 7 through 10. Note that on a preferred basis, an edge to each component part includes a strip of adhesive tape AT for allowing the catheter 10 to adhere to itself after proper folding about the patient's scrotum. An additional section of adhesive tape can be included along base 20, or ATB.

For detailed directions, the catheter 10 first folds winged apron-shaped base 20 upward (per Arrow A1 in FIG. 7); followed by the first side-to-side wrap around of shorter first wing end 14 (in the right-to-left direction of Arrow A2 in FIG. 8); then sequentially ending with the flapping of longer second wing end 16 (in the left-to-right direction of Arrow A3 in FIG. 9) to form the fully folded catheter unit 10 of FIG. 10.

FIG. 8 is a top plan view showing the order of folding per installation step 1;

FIG. 9 is a top plan view showing the order of folding per installation step 2; and

FIG. 10 is a top plan view showing the order of folding per installation step 3.

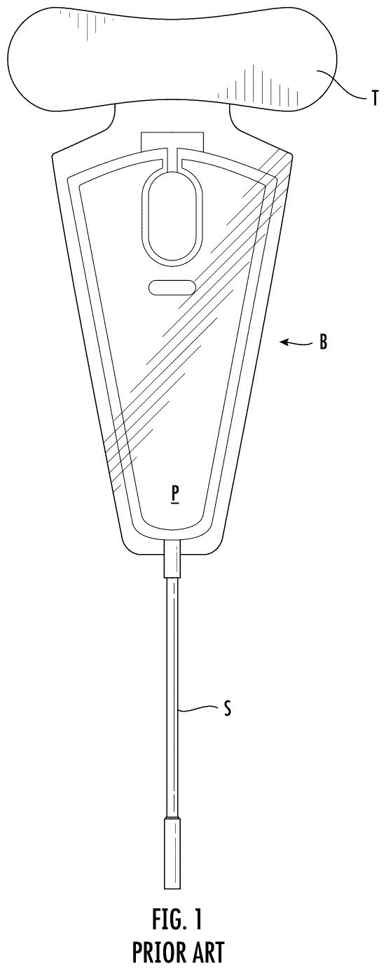

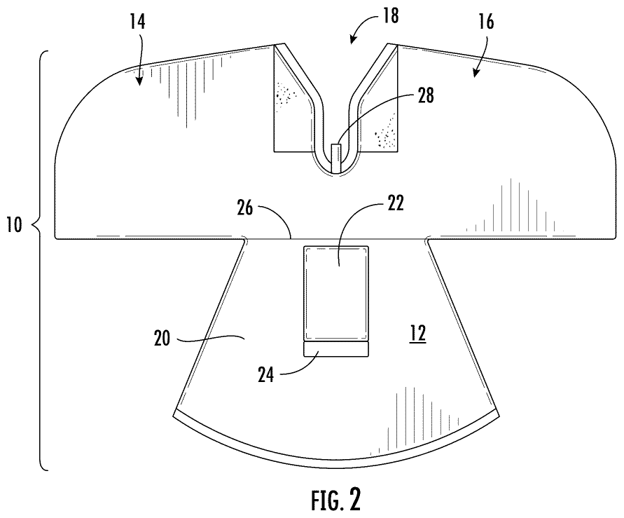

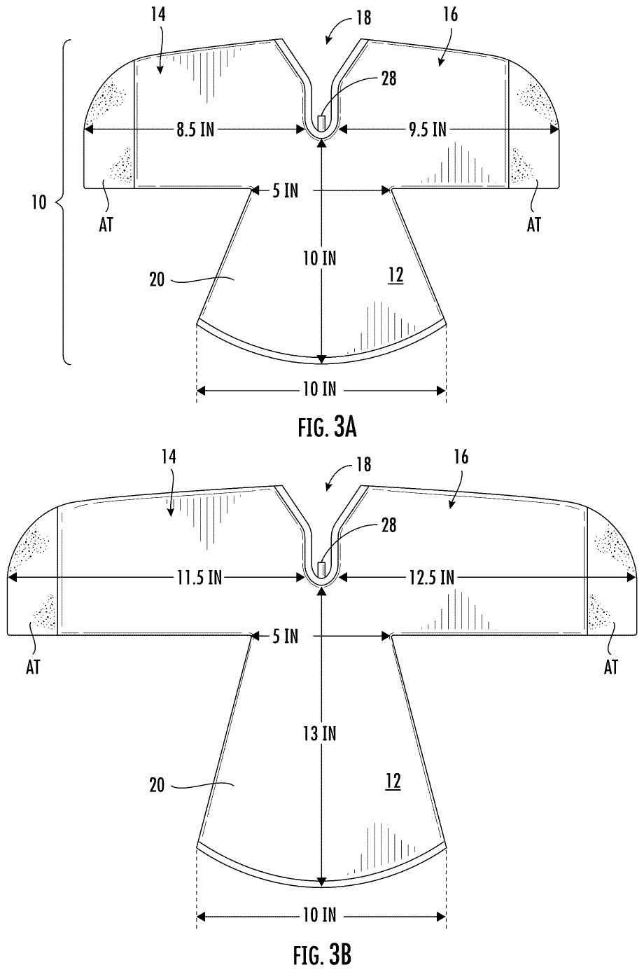

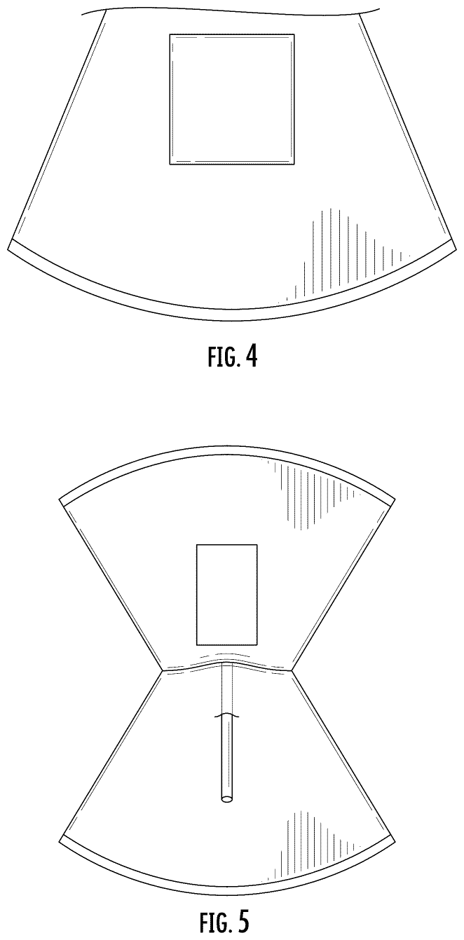

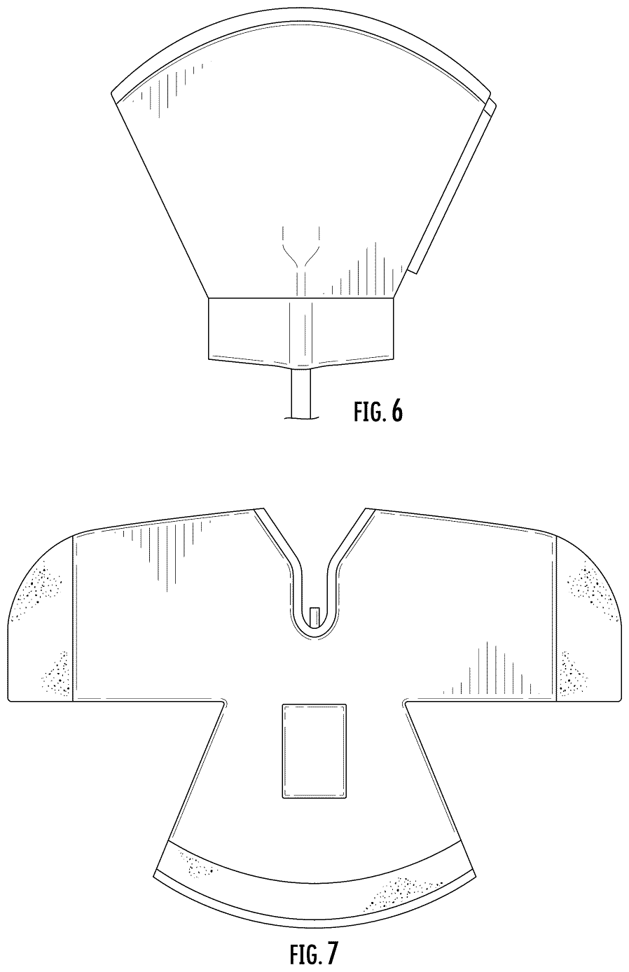

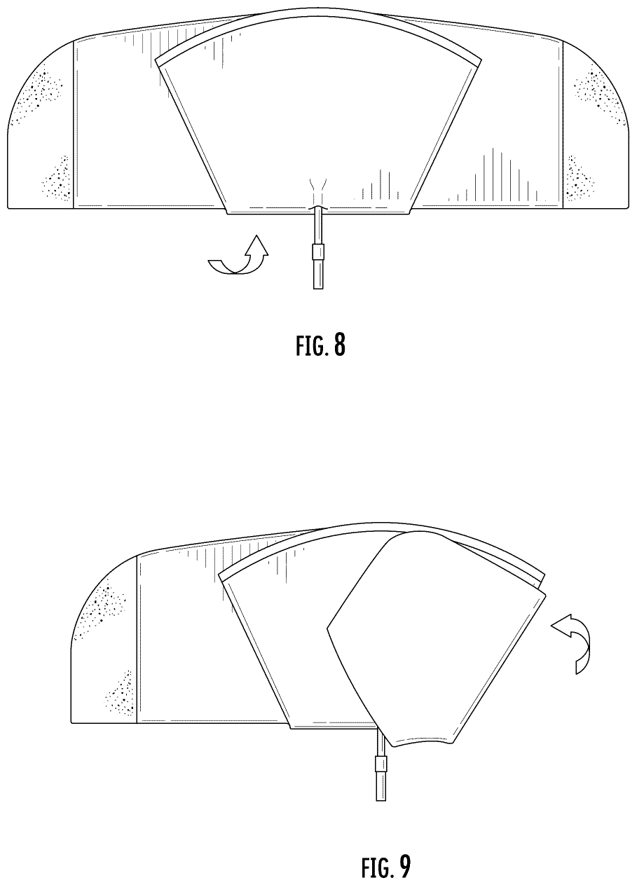

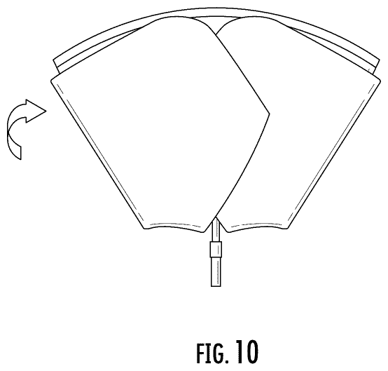

It should be further noted that the fully assembled unit of FIG. 10 may be secured against the representative patient's thighs (or diaper interior) so long as the suction tubing connection can be accessed through the bottom edge of the unit-as gravity flow, downward, is still a key component to the day-to-day functioning of this invention.

With regard to preferred apertures in the suction tubing proper, it should be noted that one representative embodiment includes a diameter with entrance points at a 3.3 cm mark. A subsequent variation uses a 6 mm diameter aperture configuration. Furthermore, though not shown in the accompanying FIGS., it is to be understood that an elongate tube with a plurality of suction apertures may be replaced with alternate variations of urine collection tube shapes, such as a T-shaped version, not shown.

### DETAILED DESCRIPTION OF THE INVENTION

As stated above, many male patients are not able to use the currently made and sold model of male external catheter due to issues with size or retraction of their penises. Current apparatus that use a Ziploc® bag type configuration in a funnel shape may achieve efficacy depending on relative sizes AND on hopes that a urine collector bag will not accidentally fold over on itself. This invention obviates such prior art concerns.

With the foldable “gauze” pad having an internal (preferably integral) suction catheter, the risks of a patient soiling himself are reduced if not fully eliminated thereby further reducing risks of moisture exposure and patient skin issues. This would also be a ‘cleaner’ option because when the bag ‘folds’ on today's male external catheters, it becomes a pocket for urine to collect and back up into the urethra causing UTI concerns.

The claimed invention differs from what currently exists. As previously discussed, this invention should reduce exposure to moisture. This would also be more effective for retracted men and/or those having smaller penis sizes. Today's model of male external catheter is only useful for a small population of men. When nurses or medical personnel use such devices, they are known to fail resulting in total bed saturation and moisture exposure.

One Embodiment of this Invention comprises:
1. A moisture absorbing pad of varying sizes and shapes. One representative absorbent pad is somewhat cross-shaped though other configurations may be substituted therefor. Alternately, the main elements resemble a holiday angel costume shirt-with a lower apron between a pair of puffy sleeves having a centrally located notched region. The size and thicknesses of pads may vary, from standard to large, depending on the male patient's body frame. It is also desired that the varying layers of this catheter system be made from triple thick components—for enhanced adsorption. In usage, such pads could become expandable to anywhere ranging from 3.5 in. to 5.5 in. thick.

A primary component of this invention should include at least one side that would not leak—i.e., have an inside layer similar to that of a specialty moisture wick pad, i.e., largely absorbent with an ability to wick moisture away. It would be composed of a relatively thin mesh fabric layer, and a stiffer water-resistant sponge pad with a fenestrated tube below the sponge layer.
2. The “cross-shaped pad should be placed with the notched arch of the cross beneath where the shaft of the patient's penis or perineal area would be.
3. The suction catheter should be woven into the layers of the padding, about 1 to 1.5 inches below the curve of the moisture absorbing pad.
4. For a typical catheter installation, first fold the apron-based bottom side up. Next, fold the rounded sides around the penis with first folding the bottom section, then folding the sides up as numbered in FIGS. 8-10 making sure the lower side of the assembled unit (with suction tubing extending from its lowermost edge is adhered last.
5. The catheter itself can be kept adhered together for securing to the patient's upper thigh. Alternately, the assembled unit can be secured within a disposable brief.
6. Frequency of change would be either hospital policy for external catheter use or as necessary. Suction would need to be only as high as hospital policy dictates.

### How the Invention Works

Utilizing more moisture absorbent textiles and essentially wrapping the entire penis and enclosing it in a suctioned area with 360 degrees of moisture-wicking capabilities without risk of kinking, dislodging or product failure. Through using a typical ‘pad’ that is additionally used in briefing older or obese men, this helps eliminate multiple brief changes and soiled bedding concerns while reducing the risk of infection and excessive moisture exposure. The padding can sit around the base of the shaft or scrotum where the penis is. The suction catheter should remain in an area where urine may likely ‘pool’ and thereby go to the wall suction. Securement would be with existing brief to help manage stool incontinence or can be secured with adhesive pads.

### How to Make the Invention

Step A—an absorbent pad of varying sizes and various textures and suction inside the pad. Wrap pad around the patient's penis making sure that the suction catheter is situated in an area where urine is most likely to pool.
Step B—attach to wall suction and change every 12 hours.
Step C—moisture wick padding, suction catheter with an aerated ability. Then employ exterior tubing connections—especially with typical hospital grade wall suction.

### How to Use the Invention

I. reduce moisture exposure,
II. reduce need for bed changes,
III. reduce risk of infection,
IV. reduce risk of skin breakdown.

### Further Proposed Improvements Under Consideration

One of the goals of this device to enable multi-directional positioning . . . especially when intending to add internal suctioning to it. Internal to the male pad, there is a mesh fabric which would sit on, or in proximity to, the patient's urethra. Below that would be a moisture resistant, fine sponge that will allow for the flow of urine to the patient's catheter. Finally, fenestrated tubing would sit below those layers for connecting to suction. That tubing could sit between the patient's thighs with the padding folded up over his scrotum/penis for the system to still use gravity to best keep the patient dry.

Having described the best modes currently known for practicing this system and method, it is to be understood that the scope of this invention may be further described by the attached claims.

### Sequence Listing

Not applicable.

1. An external male catheter comprising a multi-layered, cross-shaped pad component into which is included a suction tubing for positioning in proximity to a patient's urethra, said pad component adapted for being folded around the patient's penis and then secured to itself.

2. The male catheter of claim 1 wherein said pad component includes a first wing end, a second wing end, a notched recess between the first and second wing end and an apron-shaped base below the first and second wing ends.

3. The male catheter of claim 2 wherein the apron-shaped base includes a mesh fabric component.

4. The male catheter of claim 3 wherein the apron-shaped base further includes an aerated sponge adjacent the mesh fabric adapted for connecting to the suction tubing.

5. The male catheter of claim 2 wherein the first wing end and the second wing end differ in relative length so that a longer wing end can overlap a shorter wing end when installing the male catheter about the patient's penis.

6. The male catheter of claim 2 wherein the first wing end and the second wing end each include adhesive tape means about a perimeter edge for securing to one another.

7. The male catheter of claim 2 wherein the apron-shaped base includes adhesive tape means along at least one perimeter edge.

8. The male catheter of claim 2 wherein said pad component includes an upper wing layer, a middle wing layer and a bottom wing layer, each consisting of a plurality of padding to create a padding pocket for the patient's penis.

9. An external male catheter comprising a multi-layered, cross-shaped pad component that includes a first wing end, a second wing end, a notched recess between the first and second wing end and an apron-shaped base below the first and second wing ends into which is included a suction tubing for positioning in proximity to a patient's urethra, said pad component adapted for being folded around the patient's penis and then secured to itself.

10. The male catheter of claim 9 wherein the apron-shaped base includes at least one of: a mesh fabric component and an aerated sponge adapted for connecting to suction tubing.

11. The male catheter of claim 9 wherein the first wing end and the second wing end differ in relative length so that a longer wing end can overlap a shorter wing end when installing the male catheter about the patient's penis.

12. A method for securing a male patient's member into a wickable catheter system, said method comprising:
(a) providing a catheter device including a cross-shaped pad and at least one section of suction tubing woven into the cross-shaped pad;
(b) positioning the male patient's member inside the cross-shaped pad of the catheter device;
(c) wrapping the cross-shaped pad about the male patient's penis, scrotum or both; and
(d) securing the wrapped penis and scrotum to an area of the male patient selected from the male patient's stomach and an upper thigh.

13. The method of claim 12 which further comprises:
(e) activating the suction tubing of the wickable catheter system.

14. The method of claim 12 wherein step (d) includes securing the wrapped penis and/or scrotum utilizing incontinence briefs.

15. The method of claim 12 wherein the cross shaped pad is sized for a standard, juvenile or bariatric use.

16. The method of claim 12 wherein the section of suction tubing includes at least one aperture at the entrance point having a diameter of less than about 10 mm.

17. The method of claim 16 wherein the aperture at the entrance point has a diameter of about 6 mm or less.
